(19) United States
(12) Patent Application Publication
(10) Pub. No.: US 2021/0319717 A1
(43) Pub. Date: Oct. 14, 2021

(54) A SURGICAL SIMULATION ARRANGEMENT

(71) Applicant: FOLLOU AB, Torslanda (SE)

(72) Inventor: Fredrik OLSSON, Torslanda (SE)

(73) Assignee: FOLLOU AB, Torslanda (SE)

(21) Appl. No.: 17/059,835

(22) PCT Filed: May 15, 2019
(86) PCT No.: PCT/EP2019/062490

§ 371 Date: Apr. 13, 2021

(30) Foreign Application Priority Data
May 31, 2018 (SE) 1850656-8

(51) Int. Cl. G09B 23/28; A61B 90/00; A61B 90/92; A61B 90/98

(52) U.S. Cl. CPC G09B 23/285; A61B 90/98; A61B 90/92; A61B 90/36

(57) ABSTRACT

The present disclosure relates to an arrangement for automatically identifying which simulated instrument is used in a user interface device.

### TECHNICAL FIELD

The present disclosure relates to an arrangement for automatically identifying which simulated instrument is used in a user interface device.

### BACKGROUND

Surgical simulation systems are being more and more used to train a physician in different surgical procedures in a risk-free environment. In particular, in the field of minimal invasive surgery, such as e.g. laparoscopy, arthroscopy etc. the surgical simulation systems have gained a high degree of acceptance. The simulation software has become realistic to such extent that the computer-generated images and the behavior during interaction with the simulator gives a high degree of realism, but there are still elements in the simulation significantly different from reality, and the intention of the present disclosure is to address one of them which is related to the user selection of simulated instruments.

A simulation system typically comprises a computer with simulation software, one or many user interface devices, one or many surgical instrument representations where a simulated scope with a camera is often one of them, and at least one screen that shows the simulated camera. The simulation system makes up an advanced computer game in which the user can play and learn surgical skills and surgical procedures in a safe environment, and therefore becomes a realistic and effective training environment. A simulation instrument consists of a physical instrument representation and a virtual instrument representation. The physical instrument representation is what the user holds in his hand and resembles a real surgical tool but doesn't necessarily have to look exactly the same as the real surgical tool it intends to simulate. The virtual instrument representation is the visual appearance and a behavior model and is often modeled with the highest possible fidelity to match the corresponding real instrument. The user will see the visual appearance of the instrument on the screen and interact with the simulated environment, such as anatomies and tissues, according to the behavior model.

As a part of a simulation exercise, a user can pick an instrument (meaning the physical representation of it) and insert it into a user interface device, which then tracks the movements of the instrument. These movements are sent to the simulation software, which simulates a visual and physical response, such as position and orientation of all instrument, opening and closing of grasper type instruments, collisions and interactions with anatomies and tissues resulting in model deformations. Some user interface devices have force-feedback capability and then the physical response from the user interaction is sent back to the interface device, which then applies forces and torques that corresponds to the physical response. This gives the user the sensation that he or she touches e.g. tissues or anatomies in the exercise.

In some of the prior-art solutions for interface devices, an instrument representation is an integral part of the interface device, meaning that the instrument cannot be pulled out of the interface device, because it is prevented mechanically to do so. In other prior-art solutions for the interface devices, it is possible to pull out the instrument representation, put it away, pick it up again and insert it back into the user interface device. Since an exercise of a surgical procedure often involves usage of several different type of instruments, and switching between those types during the procedure, the simulation has to be informed about which instrument that is currently selected. The instrument selection method in the prior-art solutions is that the user tells the simulation program via a graphical and/or electromechanical user interface. It can for instance be a selection on a touch screen, on a keyboard, by pressing a pedal repeatedly or by opening or closing a surgical handle repeatedly until the desired instrument is selected. This selection method is used in all known prior-art simulation systems regardless of how the interface device and the instrument representation is arranged, i.e. regardless of if the instrument can be pulled out of the device, or not.

One example on the need for switching instruments is very common and relates to many surgical procedures, e.g. laparoscopic appendectomy (where appendix is removed with minimal invasive surgery) is that the surgeon, in a real procedure, is alternately handling a bipolar forceps and a scissor. The bipolar forceps is a kind of electropolar tool used to accomplish hemostasis in a section of a tissue, and the scissor is used to then cut that section. The alternation of the two surgical instruments continues until a complete and intended part of the tissue is cut away. So, the user will switch tools many times just for this specific part of the procedure. For most real procedures there will be many instrument changes along the procedure. This is something that the corresponding simulated procedure also takes into account, by letting the user select the appropriate simulated instrument throughout the procedure exercise. By using the prior-art methods to tell the simulation which instrument that is chosen, the selection introduces an unrealistic step that prevents him or her to follow a correct behavior for the instrument handling. A common way to make the selection in the prior-art simulators is to pull back the instrument fully (but not out) and then scrolling among a selection of instruments presented on a display by opening and closing the handle until a desired instrument is highlighted, and finally pressing a pedal to confirm the selection. In fact, this selection method is often much easier than in reality, because in reality the surgeon pulls out one instrument, while still manipulating a tissue or an organ with the other instrument inside e.g. the abdomen, then reaches for the next desired instrument, then inserting this instrument into a port that may have changed its orientation and then finding back with the instrument to the target area. This “withdrawal and insertion” exercise is an important and difficult skill to train on for the trainee, and is completely omitted in prior-art simulation systems.

Another aspect of the current prior-art instrument selection methods is that the selection of the instrument doesn't involve any other person than the user standing in front of the simulation station. In reality, other persons in the surgical team are involved in the selection of instruments, and therefore is this part of the training of a surgical team not possible to do in a realistic manner. This part is also important to train, since it requires clear and predictive information exchange within the team.

Accordingly, although the existing surgical simulators are quite well suited for individual training they still lack in realism in the abovementioned aspects, which opens for further improvements that can make both individual and team training more realistic and thereby provide a more powerful educational platform, that effectively can further mitigate the risk for errors in the real operating room. Thus, there seems to be room for further improvements in relation to the handling and selection of surgical simulation instruments in simulated exercises.

### SUMMARY

It is an objective of the present disclosure to address the limitations of the prior art, and to provide a more natural handling and selection of surgical simulation instruments, which gives a basis for an improved educational platform, by an extended and improved functionality.

According to an aspect of the present disclosure, the above is at least partly met by a surgical simulation arrangement, comprising a simulation instrument representation, an instrument receiving device, the instrument receiving device comprising means for detachably receiving the simulation instrument representation, an identification unit, a display unit, and a control unit connected to the instrument receiving device, the identification unit and the display unit, wherein the control unit is adapted to receive, from the identification unit, an indication of a mating between the instrument receiving device and the simulation instrument representation, the indication comprising identifiable information for the simulation instrument representation, and display, at the display unit, a depiction of the simulation instrument representation based on the identifiable information and in relation to the instrument receiving device

In line with the present disclosure, the surgical simulation arrangement may comprise one or a plurality of physical instrument representations (hereby referred to as “instruments”), one or a plurality of user interface devices, a computer with simulation software, a screen and one or a plurality of identification units. The user interface device may accordingly be arranged to receive an instrument detachably, meaning that the instrument can be inserted into and withdrawn out from a user interface device. Each user interface device typically has a physical position, corresponding to e.g. a port on a simulated patient, and the collection of user interface devices makes up the “user interface device setup”. Each user interface device may also have a virtual position, which may or may not be the same as the physical position.

The virtual positions are used in the simulation as port positions on a virtual patient. The identification unit is arranged to provide information about which instrument is inserted or is intended to be inserted into which user interface device. In other words, the identification unit may provide information about an existing or intended “mating” between one of the instruments and one of the user interface devices. The information about a mating, coming from an identification unit, may be used in the simulation program to present a virtual (visual) representation of the instrument, positioned according to information about the user interface device virtual position and oriented and manipulated according to movement data from the mated user interface device.

As a first alternative (alternative A), an identification unit is arranged in or on a user interface device (in principle there will be one identification unit per user interface device) and where it is arranged to read identifiable information from an instrument (the “identity” of the instrument) that is inserted into or is in a resolvably close vicinity to the user interface device. The mating information is complete because the identification unit is tied to the user interface device, and the instrument identity is detected by that identification unit.

As a second alternative (alternative B), an identification unit is arranged in or on an instrument (in principle there will be one identification unit per instrument) and where it is arranged to read identifiable information from a user interface device (the “identity” of the user interface device) when the instrument is inserted into or is in a resolvably close vicinity to a user interface device. The mating information is complete because the identification unit is tied to the instrument, and the user interface device identity is detected by that identification unit.

As a third alternative (alternative C), an identification unit is arranged in a close vicinity to the simulation system (there can be one or a few identification units close to the simulator) and where it is arranged to read the identity of an instrument, by letting the user approach an instrument to the identification unit. The instrument is thereby “scanned” and the identification unit holds this information until another instrument is scanned. The mating will be complete when the scanned instrument is inserted into a user interface device either by having a separate instrument detector (that detects the presence of an instrument) or by analyzing a movement in the user interface device, e.g. the instrument translational movement.

As a fourth alternative (alternative D), an identification unit is arranged in an instrument stand and is arranged to detect when an instrument is removed from or put back into the stand. The instruments are organized in a specific order in the stand. The information about which instrument is selected by the user is determined by the latest removed instrument position in the stand and the predetermined organization of the instruments. As in the third alternative, the mating will be complete when the scanned instrument is inserted into a user interface device either by having a separate instrument detector (that detects the presence of an instrument) or by analyzing a movement in the user interface device, e.g. the instrument translational movement.

The present disclosure solves an automatic identification and natural selection of instrument, which has not been made in existing solutions, and this opens up the new and improved features in simulation based surgical training, as described above.

Further features of, and advantages with, the present disclosure will become apparent when studying the appended claims and the following description. The skilled addressee realize that different features of the present disclosure may be combined to create embodiments other than those described in the following, without departing from the scope of the present disclosure.

### BRIEF DESCRIPTION OF THE DRAWINGS

The various aspects of the present disclosure, including its particular features and advantages, will be readily understood from the following detailed description and the accompanying drawings, in which:

FIG. 1 is a schematic view of a surgical simulation system arranged to automatically identify a selected instrument;

FIG. 2a illustrates a simulation system with identification units according to said first alternative;

FIG. 2b illustrates a simulation system with identification units according to said second alternative;

FIG. 2c illustrates a simulation system with and identification unit according to said third alternative;

FIG. 2d illustrates a simulation system with and identification unit according to said fourth alternative;

FIG. 3 illustrates details of an identification unit according to a preferred embodiment of the present disclosure;

FIG. 4 illustrates further details of an identification unit according to a preferred embodiment of the present disclosure.

### DETAILED DESCRIPTION

The present disclosure will now be described more fully hereinafter with reference to the accompanying drawings, in which different alternatives for embodiments and the currently preferred embodiments of the present disclosure are shown. This present disclosure may, however, be embodied in many different forms and should not be construed as limited to the embodiments set forth herein; rather, these embodiments are provided for thoroughness and completeness, and fully convey the scope of the present disclosure to the skilled addressee. Like reference characters refer to like elements throughout.

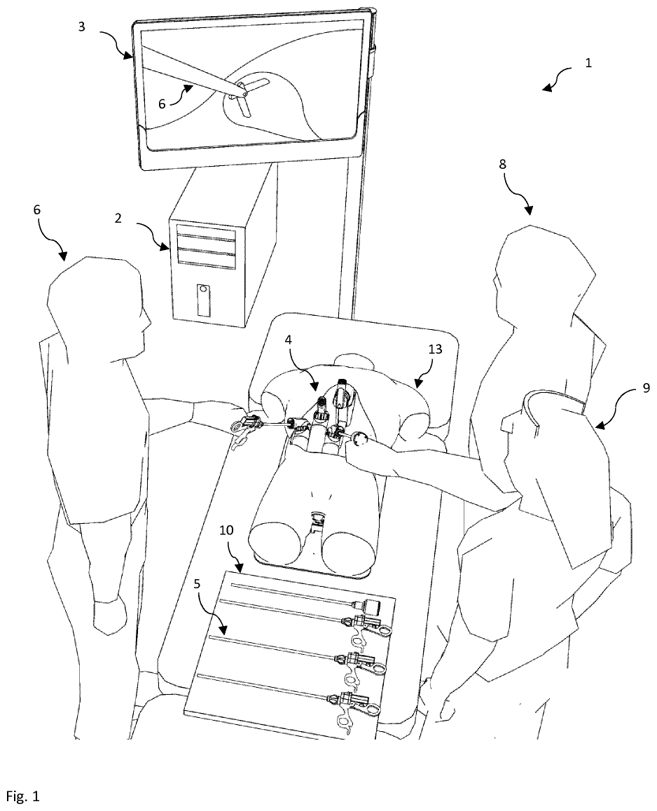

With reference to FIG. 1, the simulation system (1) comprises a control unit (2) running simulator software for simulating a surgical procedure, and a display (3) for displaying a visualization of the simulated procedure to the user or users (6, 8, 9). One or a plurality of user interface devices (4) is connected to the control unit (2), and the user interface devices are arranged to provide manipulation input to the control unit (2), thereby letting the user interact with the simulation. A user interface device (4) has a physical position that is often related to a physical representation of a patient representation, it can e.g. be a manikin, a torso (13), a limb or a part of a simulation working station. The user interface device (4) also has a corresponding virtual position, which relates to the virtual representation of the patient, it can e.g. be a portal position in the abdomen. The simulation system further comprises one or a plurality of instruments (5) which retractably can be connected with a user interface device (4), meaning that the instruments can be inserted into and withdrawn from the user interface device (4). The instrument comprises a handle portion (5a) and an elongated portion (5b) that can be inserted into a user interface device (4). The handle portion (5a) can be a real handle used in surgical procedures, or it can be a mockup of a real handle. Any kind of handle for the applicable surgical procedures can be mounted on the elongated portion (5b), such as, but not limited to, a grasper, a scissor, a clip applier, a forceps, a laparoscope etc. The instrument handle (5a) often has an additional degree of freedom for the user such as a grip portion for a scissor-like handles or a turning motion of the laparoscope camera (not depicted here). The additional degree of freedom for a handle used in a simulator is tracked with a sensor. Furthermore, the handle can be equipped with an actuator to provide force feedback. Neither the tracking of the handle nor the force feedback mechanism is described further in this context but is only mentioned as an orientation in the art of surgical simulation.

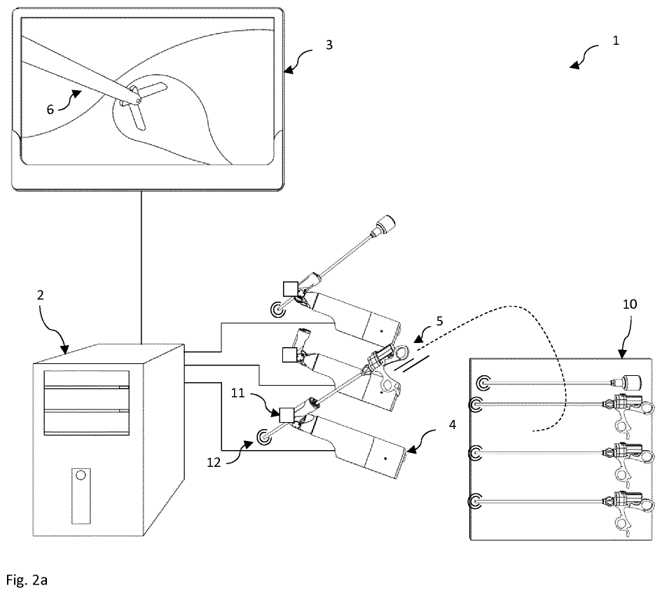

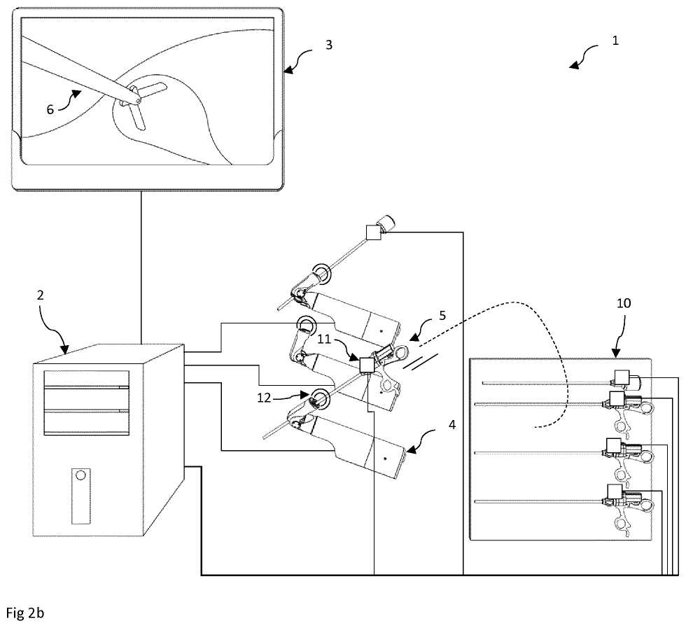

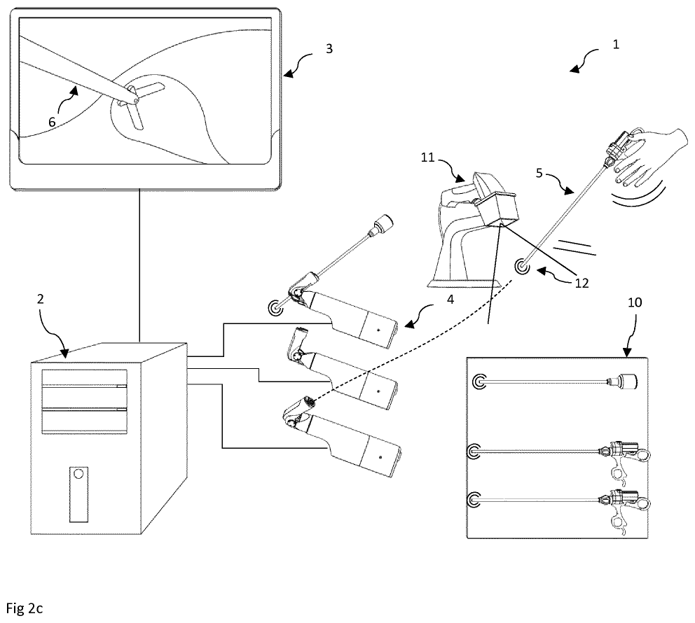

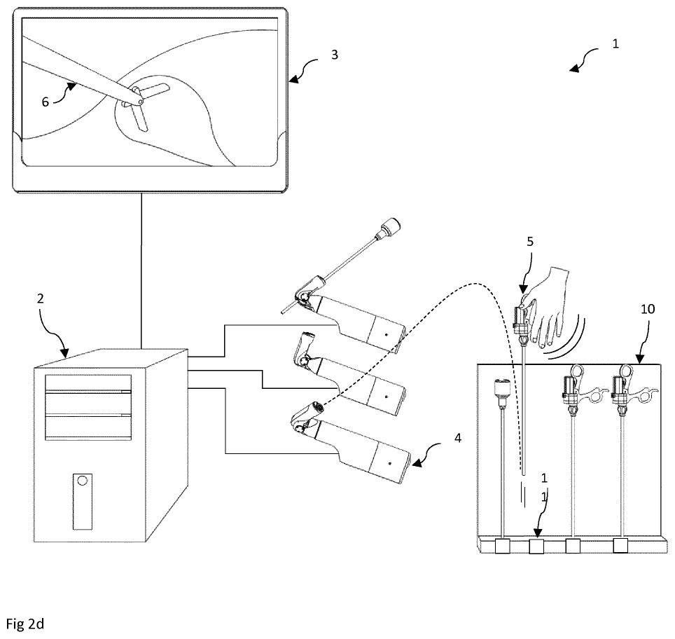

The user can select an instrument (5) from a set of instruments (10), where the instruments represent real instruments, each having a virtual instrument representation (6) with a visual model and a behavioral model in the simulation. An identification unit (not depicted in FIG. 1 but in FIGS. 2a, 2b, 2c, 2d, 3 and 4) is arranged to automatically provide information about which instrument the user selected and in which user interface device the selected instrument is inserted. The control unit (2) uses the selection information to visualize, on the display (3), and simulate the corresponding virtual instrument representation (6) of the selected instrument with interaction input from the user interface in which the instrument was inserted into, where the corresponding user interface device virtual positioning is used as a positional reference in the simulation.

Four principally different implementation alternatives (same as mentioned in the summary) for identifying a selected instrument are disclosed below.

Alternative A: With reference to FIG. 2a, the instrument (5) carries identifiable information (12) and each user interface device (4) has an identification unit (11) in, on or as a part of the user interface device (4) that identifies an instrument that is being inserted into it by reading the identifiable information (12). The selected instrument (5) and the user interface device (4) are immediately mated, because the control unit holds information about which user interface device the identification unit belongs to and which instrument the identification unit identified. The identifiable information (12) can be seen as carried physically by a “tag” and the identification unit (11) is arranged to read the tag. One preferred embodiment of such arrangement is disclosed further below.

Alternative B: With reference to FIG. 2b, the user interface device (4) carries identifiable information (12) and each instrument has an identification unit (11) that identifies the user interface device it is being inserted into by reading the identifiable information. The selected instrument (5) and the user interface device (4) are immediately mated, because the control unit holds information about which instrument the identification unit belongs to and which user interface device (4) the identification unit identified.

Alternative C: With reference to FIG. 2c, the instrument carries identifiable information and a separate identification unit reads the identifiable information when the user presents the instrument to the identification unit by, e.g. approaching the identification unit with the instrument. The identifiable information can be e.g. a bar code, a RFID tag, an NFC tag, and the identification unit can be a bar code scanner, an RFID detector or an NFC detector respectively. The control unit (2) receives the identifiable information and thereby knows which instrument is selected. The user then inserts the instrument into an interface device, which detects the presence of an instrument. By letting the control unit assume that it was the latest identified instrument that was inserted into the user interface device, the mating is information complete. To detect the presence of an instrument in the user interface device, there can either be a dedicated mechanical or optical switch, or it can be information from one or a combination of many of the motion sensors in the user interface device, e.g. the sensor that tracks the longitudinal movement (in/out) of the instrument.

Alternative D: With reference to FIG. 2d, the instruments are organized e.g. in a stand (10), where the positions of the instruments are the basis for the instruments identities. The instrument stand has an identification unit that consists of one detector (11) per position that detects the presence or absence of an instrument. The user selects an instrument by picking it from the instrument stand. The identification unit provides information about the latest absent position. The control unit (2) determines the identity of the instrument by assuming that the instrument that was latest picked from the instrument stand is the instrument that was predetermined to be in that stand position. The user then inserts the instrument into an interface device, which detects the presence of an instrument. By letting the control unit assume that it was the latest identified instrument that was inserted into the user interface device, the mating is information complete. The presence of an instrument can be detected e.g. by using a mechanical, optical or magnetic switch.

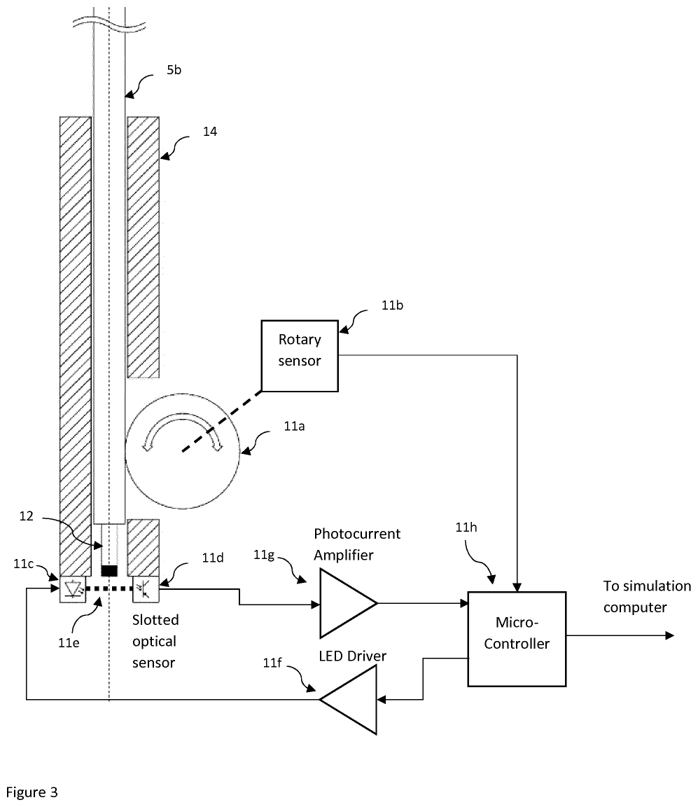

With reference to mainly FIGS. 2a, 3 and 4 a preferred embodiment of an instrument identification system is disclosed. It is implemented according to said alternative A (FIG. 2a), above, meaning that each user interface device in the system comprises an identification unit (11) and each instrument (5) carries identifiable information (12). Now with reference to FIG. 3, the identifiable information in this preferred embodiment is a tag that is a pin (12) with a unique length. The tag is fitted at the tip of the elongated portion (5b). Each instrument in a set of instruments (10, see FIG. 2a) has a tag with a unique (at least within that instrument set) tag length. The tag pin has a transparent portion and a distal opaque portion. The identification unit comprises a wheel (11a) that rotably engages to the elongated portion (5a) when the instrument is inserted some length into an instrument passage (14), which is part of the user interface device (4). When the elongated portion (5b) of the instrument (5) moves longitudinally in the instrument passage (14), the wheel (11a) rotates. The rotation of the wheel (11a) is measured with a rotary sensor (11b) which is connected to a microcontroller (11h) in the user interface device, and the rotation angle from the rotary sensor and the diameter of the wheel (11a) can be used to determine the travel length of the elongated portion (5b). A slotted optical sensor consisting of a light emitting diode (LED) (11c), an air gap and a photodetection sensor (a photoelectric diode or transistor) (11d) is fitted at the end of the instrument passage (14). The said slotted optical sensor detects if the air gap is occluded or not. The elongated portion is arranged to travel through the air gap. The opaque part of the tag, and the elongated portion (5b), which is opaque too, will occlude the air gap, but free air and the transparent part of the transparent tag will not occlude the air gap. The LED (11c) is driven by a LED driver (110, which may or may not be controlled by a microcontroller (11h). The LED driver (110 lights the LED (11c). A photocurrent amplifier (11g) amplifies and thresholds the analog signal from the photodetection diode (11d) to provide a digital signal to the microcontroller (11h), where the two digital states of that signal correspond to the air gap being occluded or not occluded. Furthermore, the wheel (11a) and the slotted optical sensor (11c, 11e) is arranged so that the wheel engages the elongated portion before the opaque tip of the longest tag reaches the air gap in the slotted optical sensor. This ensures that the travel length of the instrument is measured before the opaque part of the tag reaches the air gap.

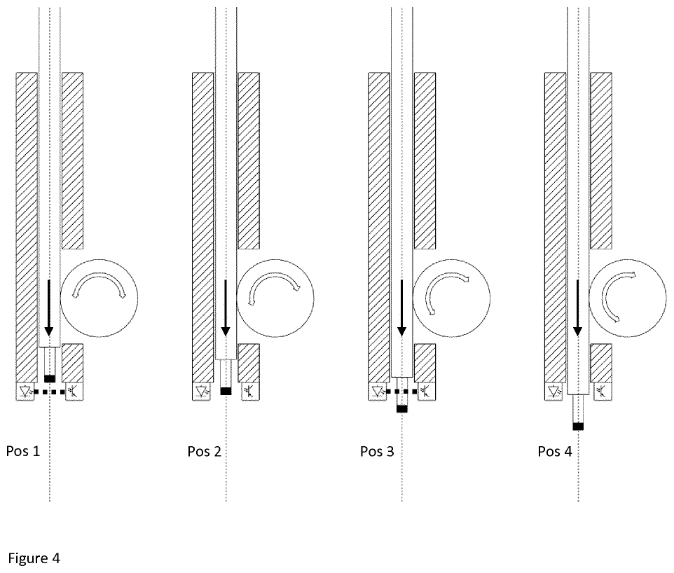

Now with reference to FIG. 4, the details of the measurement of the tag is explained. The user inserts the instrument (5) into the instrument passage (14) and the instrument elongated portion (5a) engages the wheel (11a) according to Pos 1 in FIG. 4. The air gap is in this position not occluded. The elongated portion now moves further, see Pos 2 in FIG. 4, and the opaque part of the tag occludes the air gap. The longitudinal position of the instrument is now reset to zero in the microcontroller (11h). The elongated portion now moves further, see Pos 3 in FIG. 4, and the transparent part of the tag optically opens the air gap and the air gap is therefore not occluded. This gives the microcontroller the information, together with the instrument travel length, that the opaque part of the tag has passed and the microcontroller is now standby for the next occlusion to happen. As the instrument is moved further in the instrument passage, the opaque elongated portion (5b) occludes the air gap, see Pos 4 in FIG. 4, which is registered by the microcontroller. The microcontroller can now determine the length of the tag as the current longitudinal position since the longitudinal position was reset to zero when the tip of the tag occluded the air gap. The algorithm is made robust by combining occlusion events with longitudinal distance. The identity can of the instrument can be determined by having length intervals, e.g. one interval every millimeter, so that a measure tag length of e.g. 5.7 mm is in the 5^{th} interval between 5 mm and 6 mm, and therefore the identity of the instrument is number 5. Other intervals can be chosen, and since the rotary sensor (11b) can have a very high resolution, the precision of the length measurement can be high, and therefore can many identities be set.

It is noted that variants of the described preferred embodiment can easily be implemented. One example is that the opaque portion of the tag can have a certain length, that in combination of the tag length gives a unique identity. A second example is that the opaque portion can have a certain length which is unique, and the tag length is constant or irrelevant. A third example is that the tag can be a striped pin, giving a binary code which is unique. A fourth example is that the tag can have more than one opaque portion and that the combination of lengths of those opaque portions and possibly also the transparent portions gives the unique identity. If a combination of tag lengths, opaque portion length, etc. is used, the combinatory can provide a large number of identities.

With respect to the described preferred embodiment above, it is noted that there are several other ways to implement a tag with identifiable information and an identification unit that reads the tag. The table below is intended to show examples of, but not limited to, other embodiments of the present disclosure.

| | Identifyable | |
|---|---|---|
| | information | |
| | in the tag | Identification unit reading method |
| 1 | Colored surface | Color detector + different color thresholds |
| 2 | Colored light | Color detector + different color thresholds |
| 3 | Infrared coded light | Infrared detector and decoder. |
| 4 | Pin with a specific | Photo detector + different transparency |
| | transparency/opacity | thresholds |
| 5 | Magnetic | Magnetic flux or magnetic orientation |
| 6 | Bar code | Bar code reader |
| 7 | QR code | Camera and QR-code processing |
| 8 | RFID | RFID detector |
| 9 | NFC | NFC detector |
| 10 | Other radio signal with | Other radio signal detector + decoder |
| | coded information | |
| 11 | Mechanical sequence | Mechanical reader/Magnetic reader/optical |
| | code | reader/+ decoder |
| 12 | Coded sound signal | Microphone + decoder |
| 13 | Resistance | Resistance detector + different resistance |
| | | thresholds |
| 14 | Capacitance | Capacitance detector (based on e.g. |
| | | resonance frequency or transient |
| | | response) + different capacitance |
| | | thresholds |
| 15 | Inductance | Inductance detector (based on e.g. |
| | | resonance frequency or a transient |
| | | response) + different inductance |
| | | thresholds |

With the above described automatic instrument identification solution, the user can pick up one of several instruments from a table and insert in into one of several user interface devices without explicitly telling the system first. The user interface device chosen for insertion by the user will then detect and identify instrument chosen by the user. In the simulation software (3), the information can now be used to render and simulate that specific instrument appearance and behavior without the need for an explicit selection from the user. This feature significantly improves the user's ability to interact with the system (1) in a more realistic manner. A simulation of a certain surgical procedure can be prepared by associating a number of instruments with a specific instrument identity numbers respectively. When this is done, the user doesn't need to make any instrument selections during the exercise, but only focus on picking the right instrument from a set of instruments, either according to instructions from the simulation system, or according to his or her own choice for the most suitable instrument for a particular procedure step.

Another aspect of the abovementioned instrument identification feature is that the user can train on elements of instrument handling that hasn't been possible before. One example is when the user holds a tissue with one instrument and then needs to change the second instrument during a critical phase of the procedure. One hand is then occupied with a critical task and the other hand needs to perform a retraction movement, switching instrument, and the inserting the new instrument to finally reach roughly the same region in the body without colliding and harming other organs or tissues.

The control functionality of the present disclosure may be implemented using existing computer processors, or by a special purpose computer processor for an appropriate system, incorporated for this or another purpose, or by a hardwire system. Embodiments within the scope of the present disclosure include program products comprising machine-readable medium for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a sequence the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. Additionally, even though the present disclosure has been described with reference to specific exemplifying embodiments thereof, many different alterations, modifications and the like will become apparent for those skilled in the art.

In addition, variations to the disclosed embodiments can be understood and effected by the skilled addressee in practicing the present disclosure, from a study of the drawings, the disclosure, and the appended claims. Furthermore, in the claims, the word “comprising” does not exclude other elements or steps, and the indefinite article “a” or “an” does not exclude a plurality.

1. A surgical simulation arrangement, comprising:
a simulation instrument representation,
an instrument receiving device comprising an instrument receiving portion, the instrument receiving device configured to detachably receive the simulation instrument representation,
an identification unit,
a display unit, and
a control unit connected to the instrument receiving device, the identification unit and the display unit, the control unit comprising one or more computer processors,
wherein the control unit is adapted to:
receive, from the identification unity when the simulation instrument representation is inserted at the instrument receiving portion, an indication of a mating between the instrument receiving device and the simulation instrument representation, the indication comprising identifiable information for the simulation instrument representation and a position for the instrument receiving device, the identifiable information for the simulation instrument representation being comprised with the simulation instrument representation,
determine a type of the simulation instrument representation based on the identifiable information for the simulation instrument representation, and
display, at the display unit, a depiction of the simulation instrument representation based on the determined type of the simulation instrument representation and in relation to the position of the instrument receiving device.

2. (canceled)

3. (canceled)

4. The surgical simulation arrangement according to claim 1, wherein the simulation instrument representation comprises a tag relating to the identifiable information

5. The surgical simulation arrangement according to claim 4, wherein the identification unit is comprised with the instrument receiving device.

6. The surgical simulation arrangement according to claim 4, wherein identification unit is arranged as a separate unit comprised with the surgical simulation arrangement.

7. (canceled)

8. (canceled)

9. The surgical simulation arrangement according to claim 1, further comprising an instrument stand for holding a plurality of simulation instrument representations.

10. The surgical simulation arrangement according to claim 9, wherein the instrument stand comprises means for indicating when one of the plurality of simulation instrument representations is removed from the instrument stand.

11. The surgical simulation arrangement according to claim 10, wherein the means for indicating when one of the plurality of simulation instrument representations is removed from the instrument stand includes at least one of:
a tag for each of the plurality of simulation instrument representations, the tag comprising identifiable information each of the plurality of simulation instrument representations, and
means for detecting the identifiable information for the simulation instrument representation.

12. (canceled)

13. The surgical simulation arrangement according to claim 4, wherein the tag comprises at least one of an identifiable:
colored surface,
colored light,
pin length,
pin with a specific transparency/opacity,
striped pin,
magnetic portion,
bar code,
rfid element,
nfc element,
mechanical sequence code,
coded sound signal,
resistance element,
capacitance element, and
inductance element.

14. The surgical simulation arrangement according to claim 4, wherein the means for detecting the identifiable information for the simulation instrument representation comprises at least one of:
a color detector,
a color photodetector,
a photo detector,
a magnetic flux or magnetic orientation sensor,
a bar code reader,
a rfid detector,
a nfc detector,
a magnetic or optical reader,
a microphone,
a current or voltage detector,
a capacitance detector, and
a inductance detector.
